(19) United States
(12) Patent Application Publication
(10) Pub. No.: US 2009/0202653 A1
(43) Pub. Date: Aug. 13, 2009

(54) Compositions and method for enhancing proteoglycan production

(76) Inventors: Mark W. Erwin, Toronto (CA); Paul T. Salo, Calgary (CA); Robert Inman, Toronto (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 10/472,755

(22) PCT Filed: Apr. 3, 2002
(86) PCT No.: PCT/CA02/00486

§ 371 Date: Mar. 26, 2004

Related U.S. Application Data
(60) Provisional application No. 60/280,850, filed on Apr. 3, 2001.

(51) Int. Cl. A61K 35/12; C12N 5/06

(52) U.S. Cl. 424/520; 435/404; 435/325; 435/395; 435/375

(57) ABSTRACT

The present invention provides compositions comprising notochord enriched media and/or one or more factors derived therefrom. Such compositions are useful for enhancing the production of proteoglycan in cells or animals in need thereof, for example for treating degenerative disc disease. The notochord enriched media is preferably obtained from a nonchondrodystrophic animal.

### FIELD OF THE INVENTION

The invention relates to compositions and methods for enhancing proteoglycan production. Specifically, the present invention provides a composition comprising notochord enriched media and/or factors derived from notochord enriched media, and the use of such compositions to enhance proteoglycan production in chondrocytic cells.

### BACKGROUND OF THE INVENTION

Degenerative disc disease is one of the most common causes of disability in North American society. The intervertebral disc is an avascular structure made of a sparse amount of cells interspersed in an extracellular matrix composed of mainly collagen, proteoglycan and water. During the aging process the disc experiences certain biochemical, structural and morphological changes. The effects of these changes are most significant in the nucleus pulposus, which is where many believe that disc degeneration begins. Some of the factors implicated in these changes are disc cell nutrition, degradative enzymes, inflammatory mediators, apoptosis and prolonged mechanical loading. A decrease in cell viability and changes in the matrix composition of the intervertebral disc are visible signs of degenerative disc disease that may be detected during the aging process.

Not every person develops degenerative disc disease. There is no biological explanation for the disparity in people who do and do not develop degenerative disc disease in the absence of trauma. An important observation in this regard is that some animals do not develop degenerative disc disease and it is these species that maintain a population of disc notochord cells into adult life. The canine species is a case in point with respect to factors that may have a genetic link. Nonchondrodystrophic dogs maintain their notochord cells for many years and are not known to develop degenerative disc disease, whereas other species of purebred dogs such as beagles (the chondrodystrophic breeds) do develop degenerative disc disease.

It is considered that the loss of aggregating proteoglycan and the loss of the associated water content of the nucleus leads to a loss of the resiliency of the disc and compromised load-bearing capacity. The result of such matrix degeneration is further internal derangement of the nucleus, which seems to parallel the molecular disorganization of the nucleus extracellular matrix. It has been reported that the non-aggregating proteoglycans, that seem to arise in the process of degenerative disease, lack a binding site at the hyaluronan central protein core. Therefore, the development of substances that can enhance the production of aggregating proteoglycan can lead to effective treatments for degenerative disc disease and other disorders that involve degeneration of the matrix of chondrocytic cells.

### SUMMARY OF THE INVENTION

The inventors have found that notochord enriched media and/or factors derived therefrom, stimulate proteoglycan production in bovine disc chondrocytes. As well, the inventors have verified the expression of several genes important to chondrocyte metabolism that are increased in expression as a result of culture with these factors. These genes have been shown to be active in bovine and human disc chondrocytes. Therefore, the present invention relates to a composition for enhancing the production of proteoglycan in a cell or animal in need thereof comprising notochord enriched media and/or one or more factors derived from notochord enriched media. Preferably the notochord enriched media, and/or one or more factors derived therefrom, are from a nonchondrodystrophic animal, for example, nonchondrodystrophic canines, rabbits or felines, and the cell is a chondrocytic cell, such as a chondrocyte from the disc or articula cartilage.

The present invention further involves a method for enhancing proteoglycan production comprising administering to a cell or animal in need of such treatment, an effective amount of a composition comprising notochord enriched media and/or one or more factors derived therefrom. The invention also relates to a use of a composition comprising notochord enriched media and/or one or more factors derived therefrom to enhance the production of proteoglycan in a cell or animal in need thereof, and a use of a composition comprising notochord enriched media and/or one or more factors derived therefrom to prepare a medicament to enhance the production of proteoglycan in a cell or animal in need thereof. Preferably the cell is a chondrocyte and the subject is a mammal, in particular, humans.

In an embodiment of the present invention, the cell is an intervertebral chondrocyte, therefore there is provided a method of treating degenerative disc disease comprising administering to a cell or animal in need of such treatment, an effective amount of a composition comprising notochord enriched media and/or one or more factors derived therefrom. The invention also relates to a use of a composition comprising notochord enriched media and/or one or more factors derived therefrom to treat degenerative disc disease in a cell or animal in need thereof, and a use of a composition comprising notochord enriched media and/or one or more factors derived therefrom to prepare a medicament to treat degenerative disc disease in a cell or animal in need thereof.

The present invention further relates to a pharmaceutical composition for enhancing the production of proteoglycan comprising notochord enriched media and/or one or more factors derived therefrom and a pharmaceutically acceptable carrier.

The present invention further relates to a method of preparing notochord enriched media comprising:
(a) separating a nucleus pulposus from an intervertabral disc of a nonchondrodystrophic animal to provide a total nucleus digest;
(b) separating notochord cells from the total nucleus digest; and
(c) purifying the notochord cells and culturing the notochord cells in a media to provide notochord enriched media.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a plot of proteoglycan production as a function of concentration of NCEM applied to bovine disc chondrocytes (Y-axis is counts per minute, X-axis reflects concentration of enriched media).

FIG. 2 is a plot of cell proliferation as a function of concentration of NCEM applied to disc chondrocytes as compared to DMEM only and DMEM with 10% fetal calf serum.

FIG. 3 is a Tris-glycine SDS-PAGE of notochord enriched media, stained with colloidal Coomassie blue.

FIG. 4 is a 1.7% agarose gel of PCR products from bovine disc chondrocytes cultured with NCEM; primers directed against aggrecan, versican, CD-44 and hyaluronan synthase.

FIG. 5 is a 1.7% agarose gel of PCR products from human disc cultured with NCEM; primers directed against aggrecan, versican, CD-44 and link protein

### DETAILED DESCRIPTION OF THE INVENTION

### (i) Compositions

Maintenance of intervertebral disc integrity is dependent upon the interaction of the resident cell population, notochord and chondrocyte cells, and factors produced by notochord cells play a critical role in disc structure and function by exerting an anabolic effect on intervertebral disc chondrocytes. The inventors have partially characterized the soluble anabolic factors found in canine notochord enriched media and have found that these factors, in a dose dependent relationship, up-regulate the production of disc chondrocyte proteoglycans, specifically the large, presumably aggregating species. The present inventors have also shown that several genes important to chondrocyte metabolism are increased in expression in both human and bovine disc chondrocytes that have been cultured with notochord enriched media. These proteoglycans provide the disc with an inherent ability to maintain its matrix and therefore avoid the internal disruption which otherwise proceeds inexorably with age and/or trauma.

The present invention therefore provides compositions for enhancing the production of proteoglycan in a cell or animal in need thereof comprising notochord enriched media and/or one or more factors derived from notochord enriched media.

Enhanced production of proteoglycan provides a chondrocytic cell with an ability to maintain its matrix. Accordingly, in an embodiment of the present invention, there is provided a composition for the treatment of degenerative disease of the chondrocyte matrix in a cell or animal in need thereof, comprising notochord enriched media and/or one or more factors derived from notochord enriched media. The chondrocytic cell may be any such cell, including, but not limited to intervertebral chondrocytes and chondrocytes from the articulate cartilage.

In embodiments of the present invention the chondrocyte is an intervertebral cell, the degeneration of which is a factor in degenerative disc disease. The present invention therefore provides compositions for the treatment of degenerative disc disease comprising notochord enriched media and/or one or more factors derived therefrom.

By “enhancing the production of proteoglycan” it is meant to increase, enhance or stimulate the amount of proteoglycan in the cell or animal when compared to a control. As used herein, the term “control” refers to a cell or animal under same conditions except a composition comprising notochord enriched media, and/or one or more factors derived from notochord enriched media, has not been administered thereto.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

The term “notochord enriched media” as used herein refers to media enriched in factors isolated from a notochord cell culture system. Preferably, the notochord enriched media is derived from the notochord cells of a nonchondrodystrophic animal. A nonchondrodystrophic animal is an animal which does not develop degenerative disc disease and maintains a population of disc notochord cells into adult life. Such animals include, but are not limited to, canine, rabbit or feline nonchondrodystrophic species. Preferably such animals include canine nonchondrodystrophic species.

The factors derived from notochord enriched media may be any substance which modulates the expression of proteins involved in the synthesis and/or assembly of proteoglycan in chondrocytes. Such substances may include small molecules, DNA, RNA, lipids, proteins and peptides. Preferably the factors derived from notochord enriched media are soluble anabolic proteins or peptides. Specific soluble anabolic peptides from the notochord enriched media, suitable for the compositions of the present invention, are mainly in the 25-220 kilodalton size and occupy the neutral to acidic pH range. A list of the peptides produced by notochord cells in vitro, as sequenced by mass spectroscopy, that are suspected to be involved in whole or in part with the biologic activity of NCEM is found in Table 1. Some of the main proteins identified include alpha-2-HS-glycoprotein (Fetuin), TGF-beta-receptor-lie protein and alpha fetal proteins.

The compositions of the invention are administered to cells or animals in a biologically compatible form suitable for pharmaceutical administration in vivo. By “biologically compatible form suitable for administration in vivo” is meant a form of the notochord enriched media, or factors derived therefrom, to be administered in which any toxic effects are outweighed by the therapeutic effects of the media, or factors derived therefrom. The term animal is intended to include living organisms in need of treatment for degenerative disc disease, e.g., mammals. Examples of animals include humans, canine and equine species.

The compositions comprising notochord enriched media, or factors derived therefrom, may be administered in a convenient manner such as by injection (percutaneous, subcutaneous, intravenous, etc.), oral administration inhalation, transdermal application or rectal administration. Depending on the route of administration, the active compounds may be coated in a material to protect the compounds from the action of enzymes, acids and other natural conditions which may inactive the compounds. Preferably the compositions of the invention are administered by percutaneous injection.

The compositions of the invention to be administered to a subject may further comprise an appropriate carrier, or may be co-administered with enzyme inhibitors or in an appropriate carrier such as liposomes. Accordingly, the present invention further relates to a pharmaceutical composition for enhancing the production of proteoglycan comprising notochord enriched media and/or one or more factors derived therefrom and a pharmaceutically acceptable carrier.

The term “pharmaceutically acceptable carrier” as used herein is intended to include diluents such as saline and aqueous buffer solutions. Suitable carriers are described, for example, in Remington's Pharmaceutical Sciences (Remington's Pharmaceutical Sciences, Mack Publishing Company, Easton, Pa., USA 1985).

To administer the active ingredients of the composition of the invention by other than parenteral administration, it may be necessary to coat the composition with, or co-administer the composition with, a material to prevent its inactivation. Enzyme inhibitors include pancreatic trypsin inhibitor, diisopropylfluorophosphate (DEP) and trasylol. Liposomes include water-in oil-in-water emulsions as well as conventional liposomes (Strejan et al., (1984) J. Neuroimmunol 7:27). The active compounds may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, asorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating active compounds (e.g., notochord enriched media or factors derived therefrom) in the required amount in an appropriate solvent with one or a combination of ingredients enumerated above, as required, followed by filtered sterilization. Generally, dispersions are prepared by incorporating the active compounds into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient(s) plus any additional desired ingredient from a previously sterile-filtered solution thereof.

When the active compounds (e.g., notochord enriched media or factors derived therefrom) are suitably protected, as described above, the composition may be orally administered, for example, with an inert diluent or an assimilable edible carrier. Pharmaceutically acceptable carrier includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound(s), use thereof in the therapeutic compositions is contemplated. Supplementary active compounds can also be incorporated into the compositions.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compounds (e.g., notochord enriched media or factors derived therefrom) calculated to produce the desired therapeutic effect in association with the required pharmaceutical carrier. The specification for the dosage unit forms of the invention are dictated by and directly dependent on (a) the unique characteristics of the active compounds and the particular therapeutic effect to be achieved, and (b) the limitations inherent in the art of compounding such active compounds for the therapeutic treatment of individuals. Conventional procedures and ingredients for the selection and preparation of suitable formulations are described, for example, in Remington's Pharmaceutical Sciences (1990-18th edition) and in The United States Pharmacopeia The National Formulary (USP 24 NF19) published in 1999.

The invention further contemplates compositions for enhancing the production of proteoglycan in a cell or animal in need thereof comprising one or more factors derived from notochord enriched media, for example, one or more of the peptides/proteins in Table 1. Possible proteins identified that may be useful in a composition for enhancing the production of proteoglycan in a cell or animal in need thereof include alpha-2-HS-glycoprotein (Fetuin), TGF-beta-receptor-lie protein and alpha fetal proteins. When the one or more factor derived from notochord enriched media is a protein or peptide, the protein or peptide may be modified to be more therapeutically effective or suitable. For example, the protein or peptide may be converted into pharmaceutical acceptable salts by reacting with inorganic acids including hydrochloric acid, sulphuric acid, hydrobromic acid, phosphoric acid, etc., or organic acids including formic acid, acetic acid, propionic acid, glycolic acid, lactic acid, pyruvic acid, oxalic acid, succinic acid, malic acid, tartaric acid, citric acid, benzoic acid, salicylic acid, benzenesulphonic acid, and toluenesulphonic acids. The protein or peptide may also be converted into a solvate. The term “solvate” as used herein means a protein or peptide, or a pharmaceutically acceptable salt of a protein or peptide, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The protein or peptide may also be converted into prodrugs.

The protein or peptide may be prepared using standard peptide synthesis chemistry (for example as described in “The Chemical Synthesis of Peptides” John Jones, Clarenden Press, 1991) or using recombinant DNA technology (for example as set out in Sambrook et al (Molecular Cloning: A Laboratory Manual, 2nd Ed., Cold Spring Harbor Laboratory Press, 1989; and “Current Protocols in Molecular biology, Eds. Ausubel, F M. et al. (1994) John Wiley & Son). The protein or peptide may also be isolated from the notochord enriched media.

The formation of a desired peptide or protein salt is achieved using standard techniques. For example, the neutral peptide or protein is treated with an acid in a suitable solvent and the formed salt is isolated by filtration, extraction or any other suitable method.

The formation of solvates of the protein or peptide will vary depending on the peptide or protein and the solvate. In general, solvates are formed by dissolving the protein or peptide in the appropriate solvent and isolating the solvate by cooling or using an antisolvent. The solvate is typically dried or azeotroped under ambient conditions.

Prodrugs of the protein or peptide may be conventional esters formed with available hydroxy, thiol, amino or carboxyl groups. For example, an available hydroxy, thiol, or amino may be acylated using an activated acid in the presence of a base, and optionally, in inert solvent (e.g. an acid chloride in pyridine). Also, an available “C(O)OH” group in a peptide or protein of the invention, an ester may be formed by activation of the hydroxyl group of the acid and treatment with the appropriate alcohol in the presence of a base in an inert solvent. Some common esters which have been utilized as prodrugs are phenyl esters, aliphatic (C₈-C₂₄) esters, acyloxymethyl esters, carbamates and amino acid esters.

### (ii) Method of Preparing the Notochord Enriched Media

As stated above, notochord enriched media as used herein refers to media enriched in factors isolated from a notochord cell culture system. Broadly stated, the present invention therefore provides a method of preparing notochord enriched media comprising:
(a) providing isolated notochord cells; and
(b) culturing the notochord cells in a medium suitable for maintaining the notochord cells.
   The notochord cells are preferably from a nonchondrodystrophic animal. The notochord cells may be obtained, for example, from the nucleus pulposus which is found in the intervertabral disc. The medium suitable for maintaining the notochord cells may be, for example, Dulbecco's Modified Eagle Medium (DMEM).

In an embodiment of the present invention there is provided a method of preparing notochord enriched media comprising:
(a) separating a nucleus pulposus from an intervertabral disc of a nonchondrodystrophic animal to provide a total nucleus digest;
(b) separating notochord cells from the total nucleus digest; and
(c) purifying the notochord cells and culturing the notochord cells in media to provide notochord enriched media.

In an embodiment of the present invention, the notochord enriched media is prepared as described in Example 1 herein. In this embodiment, the notochord cells are separated from the nucleus pulposus from an intervertabral disc of a nonchondrodystrophic animal using a Percoll gradient method, for example as described in Example 1. Once separated, the notochord cells must be separated from the Percoll. This may be done, for example by mixing the cells with volumes of Dulbecco's Modified Eagle Medium (DMEM) and centrifuging. This provides a pellet of cells that is pure notochord cells. The cells may then be mixed with alginate and the alginate cell solution may be treated so that it forms beads, for example by adding the solution to a solution of calcium chloride. The beads may then be washed and cultured in a medium, for example DMEM, containing one or more infection control substances, for example antibiotics and fungicides, and growth factors, such as fetal calf serum (FCS). The notochord cells (in the form of beads) may be allowed to recover for a period of time before removing the growth factors, for example by using a sodium chloride solution, before culturing in a medium, for example DMEM. This final medium is an example of a notochord enriched medium according to the invention.

Therefore, in more specific embodiments of the present invention, there is provided a method of preparing notochord enriched media comprising:
(a) separating a nucleus pulposus from an intervertabral disc of a nonchondrodystrophic animal to provide a total nucleus digest;
(b) separating notochord cells from the total nucleus digest;
(c) mixing notochord cells with alginate;
(d) converting the alginate-containing notochord cells to beads;
(e) culturing the beads on a medium comprising one or more infection control substances and growth factors;
(f) washing the beads to remove the growth factors; and
(g) reculturing beads in media to provide notochord enriched media.

### (iii) Therapeutic Methods of the Invention

The inventors have partially characterized the soluble anabolic factors found in canine notochord enriched media and have found that these factors, in the form of a composition comprising notochord enriched media, in a dose dependent relationship, up-regulate the production of disc chondrocyte proteoglycans, specifically the large, presumably aggregating species. The present inventors have further shown that several genes important to chondrocyte metabolism are increased in expression in both human and bovine disc chondrocytes that have been cultured with notochord enriched media.

Accordingly, the present invention involves a method for enhancing proteoglycan production comprising administering an effective amount of a composition comprising notochord enriched media and/or one or more factors derived therefrom, to a cell or animal in need thereof. The invention also relates to a use of a composition comprising notochord enriched media and/or one or more factors derived therefrom to enhance the production of proteoglycan in a cell or animal in need thereof, and a use of a composition comprising notochord enriched media and/or one or more factors derived therefrom to prepare a medicament to enhance the production of proteoglycan in a cell or animal in need thereof. Preferably the cell is a chondrocyte and the subject is a mammal, in particular, humans.

The term an “effective amount” or a “sufficient amount” of an agent as used herein is that amount sufficient to effect beneficial or desired results, including clinical results, and, as such, an “effective amount” depends upon the context in which it is being applied. For example, in the context of administering an agent that enhances the production of proteoglycan, an effective amount of an agent is, for example, an amount sufficient to achieve such an enhancement in proteoglycan production as compared to the response obtained without administration of the agent. An effective amount of the notochord enriched media, or factors derived therefrom, may vary according to factors such as the disease state, age, sex, and weight of the individual, and the ability of the notochord enriched media, or factors derived therefrom, to elicit a desired response in the individual. Dosage regima may be adjusted to provide the optimum therapeutic response. For example, several divided doses may be administered daily or the dose may be proportionally reduced as indicated by the exigencies of the therapeutic situation.

Enhanced production of proteoglycan provides a chondrocytic cell with an ability to maintain its matrix and therefore avoid the internal disruption which otherwise proceeds inexorably with age and/or trauma. Accordingly, in an embodiment of the present invention, there is provided a method for treating degenerative disease of the chondrocyte matrix comprising administering an effective amount of composition comprising notochord enriched media, and/or one or more factors derived from notochord enriched media, to a cell or animal in need thereof. The invention also relates to the use of composition comprising notochord enriched media, and/or one or more factors derived from notochord enriched media, to treat degenerative disease of the chondrocyte matrix in a cell or animal in need thereof, as well as the use of composition comprising notochord enriched media, and/or one or more factors derived from notochord enriched media, to prepare a medicament to treat degenerative disease of the chondrocyte matrix in a cell or animal in need thereof. The chondrocytic cell may be any such cell, including, but not limited to intervertebral chondrocytes and chondrocytes from the articulate cartilage.

In another embodiment of the present invention, the cell in need of treatment is an intervertebral chondrocyte, therefore there is provided a method for treating degenerative disc disease comprising administering to a cell or animal in need of such treatment, an effective amount of a composition comprising notochord enriched media and/or one or more factors derived therefrom. The invention also relates to a use of a composition comprising notochord enriched media and/or one or more factors derived therefrom to treat degenerative disc disease in a cell or animal in need thereof, and a use of a composition comprising notochord enriched media and/or one or more factors derived therefrom to prepare a medicament to treat degenerative disc disease in a cell or animal in need thereof.

In further embodiments, the notochord enriched media for use in the therapeutic methods of the present invention is prepared using a method as described above hereinabove (see section ii).

The present inventors have shown that the expression of several genes that are important to chondrocyte metabolism are increased in both human and bovine disc chondrocytic cells as a result of culture with a composition comprising notochord enriched media. Specifically, it has been demonstrated that the genes encoding for CD44, Hyaluronan synthase and versican are activated by notochord enriched media. Accordingly, a person skilled in the art would be able to monitor the progress of treatment with the compositions of the invention but monitoring the activity of these genes and/or proteins. A person skilled in the art could also monitor the progress of treatment with the compositions of the invention by monitoring proteoglycan production, for example, as described in Example 2, hereinbelow.

### (iv) Isolated Proteins of the Invention

The invention provides isolated proteins or peptides derived from notochord enriched media. Specifically these proteins or peptides are soluble anabolic factors isolated from notochord enriched media and/or such proteins or peptides as are produced by genetically engineered cells that will produce the active factors necessarily formed and secreted by notochord cells. The term “isolated” refers to a protein or peptide substantially free of cellular material or culture medium when produced by recombinant DNA techniques, or chemical precursors, or other chemicals, when chemically synthesized. Preferably these proteins or peptides are those proteins derived from notochord enriched media which are mainly in the 25-220 kilodalton size range and occupy the neutral to acidic pH range. A list of the peptides produced by notochord cells in vitro, as sequenced by mass spectroscopy, that are suspected to be involved in whole or in part with the biologic activity of NCEM is found in Table 1.

The proteins or peptides of the invention, derived from notochord enriched media, or isoforms or parts thereof, can either be isolated and purified from the notochord cell preparation, or, given an amino acid sequence, be prepared by chemical synthesis using techniques well known in the chemistry of proteins such as solid phase synthesis (Merrifield, 1964, J. Am. Chem. Assoc. 85:2149-2154) or synthesis in homogeneous solution (Houbenweyl, 1987, Methods of Organic Chemistry, ed. E. Wansch, Vol. 15 I and II, Thieme, Stuttgart).

Alternatively, the proteins or peptides of the invention derived from notochord enriched media, or isoforms or parts thereof, given nucleic acid sequence of the gene encoding said proteins, can be isolated by expression in a suitable host cell using techniques known in the art. Suitable host cells include prokaryotic or eukaryotic organisms or cell lines, for example, yeast, E. coli and insect cells. Recombinant expression vectors, well known in the art, can be used to express a protein derived from notochord enriched media in a host cell in order to isolate the protein. The invention provides a method of preparing an isolated protein or peptide of the invention comprising introducing into a host cell a recombinant nucleic acid encoding the protein or peptide, allowing the protein or peptide to be expressed in the host cell and isolating the protein or peptide. Preferably, the recombinant nucleic acid is a recombinant expression vector. Proteins or peptides can be isolated from a host cell expressing the protein or peptide according to standard procedures of the art, including ammonium sulfate precipitation, fractionation column chromatography (e.g. ion exchange, gel filtration, electrophoresis, affinity chromatography, etc.) and ultimately, crystallization (see generally, “Enzyme Purification and Related Techniques”, Methods in Enzymology, 22, 233-577 (1971)).

### (v) Gene Therapy

The isolated and encapsulated nature of the intervertebral disc make it an ideal site for gene transfer. This encapsulation helps to maintain high intradiscal concentrations of locally delivered vectors, while limiting potential systemic side effects. Encapsulation may also protect the vectors from the body's own immune system which may result in a prolonged therapeutic action.

Applications of gene therapy to degenerative disc disease are reported in Cassinelli, E. H. et al. (SpineLine, Jan.-Feb., 5-11, 2001), which is incorporated herein by reference. Given the nucleic acid sequences of the genes encoding the anabolic factors derived from notochord enriched media, a person skilled in the art, using the techniques described in Cassinelli et al., would be able to develop genetically engineered cells, or other vectors, to deliver the active components of notochord enriched media (or their analogs) to the living chondrocytes of the nucleus pulposus of the intervertebral disc. The nucleic acid sequences of the genes encoding the anabolic factors derived from notochord enriched media, may be determined, given the amino acid sequence of these proteins, using standard procedures.

The following non-limiting examples are illustrative of the present invention:

### EXAMPLES

### Example 1

### Isolation and Purification of Notochord Cell Cultures

Nearly 100% pure canine notochord and disc-derived chondrocyte cells (90-95% viability) were obtained from a mixed nucleus pulposus digest. The lumbar spines of suitable nonchondrodystrophic dogs were aseptically removed, and after careful dissection of soft tissues (particularly zygapophyseal joint capsules), the intervertebral discs were carefully transected. The nucleus pulposus is readily identified by its pearly gelatinous appearance and was removed from the surrounding annulus with sterile technique. The nucleus pulposus was suspended in Grey's balanced salt solution (GBSS) containing 10% penicillin-streptomycin and fungizone (PSF) and was washed three times. The nucleus pulposus cells were then removed from the matrix by a two-step enzymatic digestion as per Maldonado et al., 1992. Briefly, this technique consisted of dissection of the nucleus pulposus from the intact disc. The first enzymatic step occurred after three washes in 150 mM NaCl, when the tissues were incubated with 0.4% pronase for 90 minutes at 37° C. Thereafter, the tissue was washed three times with NaCl as above. The second step was an overnight digestion at 37° C. with 0.012% collagenase in Dulbecco's Modified Eagle Medium (DMEM) with no fetal calf serum. The next day the cells were washed three times with 150 mM NaCl and counted on a hemocytometer.

The nucleus pulposus of human intervertebral discs (removed at routine discectomy as an essential part of corrective scoliosis surgery) contain only chondrocyte cells as do the nuclei pulposi of bovine caudal discs. The bovine and human discs also lack the gelatinous pearly-gray appearance of the canine discs. The gross morphological differences between the canine discs containing notochord cells and bovine and human discs containing only chondrocytes without notochord cells were paralleled by their striking histological differences.

The notochord cells were then isolated from the total nucleus digest using a percoll density gradient method with some modifications. Essentially, the Percoll was prepared at a 10:1 ratio with 1.5 M NaCl to form a standard iso-osmotic concentration of percoll (SIP). The density gradient was then created by combining the SIP Percoll with DMEM with PSF and diluted to form 5-1 ml steps from a starting top density of 1.007 g/ml to a bottom density of 1.035 g/ml. The nucleus pulposus cells were washed with 150 mM NaCl and given a final wash in a non-enzymatic cell dissociation solution (without calcium or magnesium) (Sigma). The cells were centrifuged, and re-suspended in 1 ml of the cell dissociation solution and layered on the top of the discontinuous Percoll gradient in a 15-ml glass tube. The gradient containing the nucleus pulposus cells was centrifuged at 200 g for 15 minutes at 21° C. with no brake. After centrifugation, four distinct bands of cells appeared directly at the density interfaces with the top two layers containing the notochord cells at over 95% purity. The bottom layer and the pellet at the bottom of the tube had pure chondrocytes. The notochord cells were extracted from the top two layers (densities of 1.007 and 1.014 g/ml), and were counted using a hemocytometer. Cell viability was assessed initially after enzymatic digestion and again after cellular purification by the use of the trypan blue exclusion method.

The cells were freed from Percoll by mixing with 5× volumes of DMEM and then they were centrifuged at 500 g for 5 minutes. The pellet of cells (now pure notochord cells) was then mixed with an appropriate volume of 1.2% alginate in order to seed them at 1×10⁶ cells/ml of alginate (approx. 50,000 cells/bead). Next, the alginate-containing notochord cells was drawn into a 21 gauge needle and the alginate/cell solution was slowly added drop-wise to a solution of 102 mM CaCl₂. The alginate/cell solution immediately formed a bead upon mixing with the calcium chloride solution then was allowed to stabilize for 15 minutes. The alginate beads-containing cells were then washed three times with GBSS plus PSF. Finally, DMEM containing PSF and FCS (10%) was added to the alginate beads as the culture medium.

The notochord cells were allowed 48 hours to recover in culture with DMEM containing 10% FCS. Next, the beads were extensively washed with 150 mM NaCl (6×15 minutes) and placed back into culture with DMEM alone, with no FCS. After 24 hours, the enriched media from these ‘virgin’ cultures was collected as notochord enriched media (NCEM). Notochord cell cultures may be sustained for over thirty days when cultured with DMEM containing 10% fetal calf serum.

### Example 2

### Up-Regulation of Bovine Disc Chondrocyte Proteoglycan Production

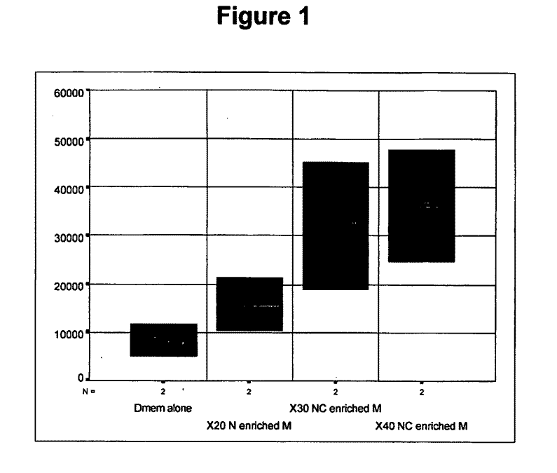

The nucleus pulposus of bovine caudal discs were removed and placed into an alginate bead culture system. After two days recovery with DMEM containing 10% fetal calf serum and antibiotics/anti-fungals, the cells were extensively washed with GBSS and exposed to a 48-hour period of culture with DMEM only. Next, the bovine chondrocytes were cultured with varying concentrations of NCEM (notochord enriched media), or with DMEM-only for four days. After the four days of culture the chondrocytes were labeled with ³⁵S (sulfate) for 18 hours followed by guanidine HCl extraction of proteoglycans and sephadex G-25 column chromatography assay of proteoglycan production. The results are given in FIG. 1, expressed as a box plot.

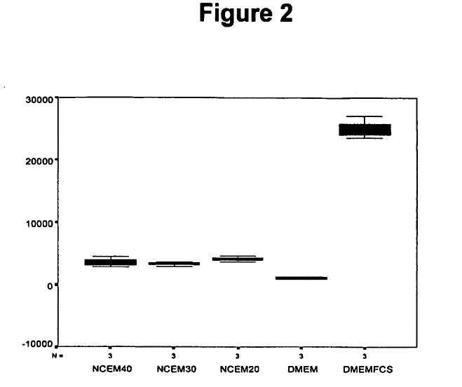

Cell proliferation, as determined by ³H thymidine labeling, as a function of concentration of NCEM applied to disc chondrocytes as compared to DMEM only and DMEM with 10% fetal calf serum was determined and the results are shown in FIG. 2. This experiment indicates that the chondrocytes have an overall four-fold increase in cell proliferation in the presence of NCEM, but this proliferation does not change much with varying concentrations of NCEM. This indicates that the increased proteoglycan production is not merely a function of increased cell numbers, and it appears that the NCEM selectively targets some aspects of the intracellular machinery that “turns on” the anabolic machinery of the cell to produce proteoglycan.

### Example 3

### Characterization of Notochord Enriched Media

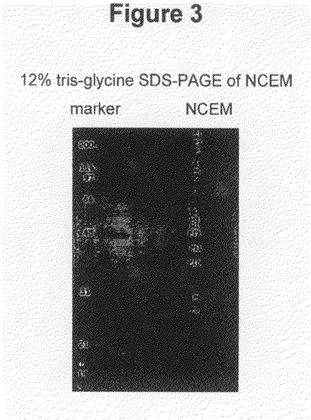

The notochord enriched media was examined by one dimensional SDS-PAGE, the results given in FIG. 3. The samples were from supernatants derived from the canine notochord cultures cultured in DMEM alone with no calf serum (see Example 1). The samples were precipitated overnight in ice cold 100% ethanol, then centrifuged and re-washed in 70% ethanol three times. Next the precipitate was solubilized in 0.04M Tris pH 6.8 and subjected to mass spectroscopy peptide sequencing. The proteins were first run on a Tris-glycine 12% SDS-PAGE gel and stained with colloidal Coomassie blue. Next the samples were trypsinized and subjected to mass spectroscopy and subsequent computer analysis comparisons of peptides detected. The SONAR™ data base/search engine was used to identify the peptide sequences. A list of the peptide/protein sequences identified using this method is found in Table 1. Some of the main proteins identified include alpha-2-HS-glycoprotein (Fetuin), TGF-beta-receptor-lie protein and alpha fetal proteins. It should be noted that, due to a lack of sequence information from the dog genome, the computer selects corresponding proteins from other species which should share a high degree of homology with the dog sequences.

The data from the SDS-PAGE were consistent from sample to sample over many cultures and indicate that there were approximately twenty-twenty five proteins produced by the notochord cells that were mainly in the 25-220 kilodalton size (see FIG. 3). These proteins, in a dose dependent relationship up-regulate the production of disc chondrocyte proteoglycans, specifically the large presumably aggregating species.

### Example 4

### RT-PCR Analysis of Total RNA Extract from Bovine Chondrocytes

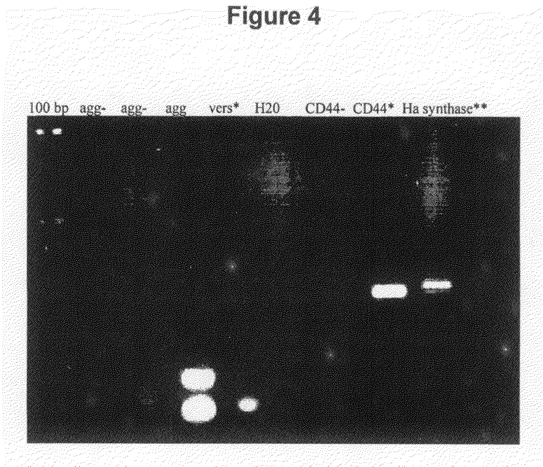

In order to determine the important genes activated by NCEM, RT-PCR analysis was performed of total RNA extracted from bovine chondrocytes cultured with NCEM. Briefly, primers were designed to probe for aggrecan and versican (the large aggregating proteoglycans), the CD44 receptor, and hyaluronan synthase. The CD44 receptor is known to assemble aggrecan at the cell surface, hyaluronan synthase is an enzyme essential to the synthesis of hyaluronic acid, the long chain polymer to which proteoglycans are attached via link proteins. It has been demonstrated that CD44, hyaluronan synthase and versican are activated by NCEM (FIG. 4). The aggrecan gene is thought to be up-regulated as well.

### Example 5

### RT-PCR Analysis of Total RNA Extract from Human Chondrocytes

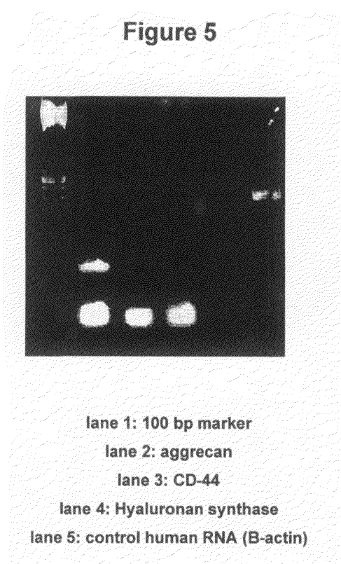

Human disc nucleus pulposus were obtained at surgery for scoliosis. The patient was a 29 year old female. The discs were from T10-L3 and were otherwise normal. The disc nucleus was obtained fresh at surgery and placed into ice cold 150 mM NaCl and copiously washed. Next the tissue was digested exactly the same as bovine disc, that is, sequential enzymatic digestion with 0.4% Pronase for 90 minutes (37 degrees C.) followed by 16 hours at 37 degrees with 0.012% collagenase, all in DMEM with 10% fetal calf serum. The next day the cells were counted with a hemocytometer, assessed by Trypan blue for viability and placed in alginate beads as per normal. The cells were allowed to recover for 48 hours and were then copiously washed with 150 mM NaCl 6×15 minutes and “starved” in DMEM only for 24 hours. Next, the cells were cultured in fresh NCEM obtained from canine notochord cultures that had been extensively washed and the NCEM collected as per normal AFTER the washes and 24 hours in DMEM alone. The human chondrocytes were cultured in NCEM for four days after which the cells were freed from the alginate by 55 mM sodium citrate in 150 mM NaCl, washed three times and then the total RNA extracted with Trizol™. The total RNA was then reverse transcribed and then amplified using the human primers for aggrecan, CD-44, hyaluronan synthase and link protein. The result, shown in FIG. 5, indicated that genes encoding for aggrecan, CD-44 and Hyaluronan synthase were upregulated.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Mass spectroscopy identified peptides contained within NCEM

Sample F1 & G1 Pooled
1. 0.82 (nr-Homo-sapiens) 36.0 kDa-gi|14743331|ref|XP_002298.2| TGF beta
receptor associated protein-1 [Homo sapiens]
Sample F2 & G2 Pooled
1. 8.8 × 10 − 8
(nr-Other-Mammalia) 67.8 kDa-gi|3319897|emb|CAA76841.1| (Y17737) albumin
[Canis familiaris]
4. 2.6 × 10 − 7
(nr-Other-Mammalia) 70.6 kDa-gi|1351908|sp|P49064|ALBU_FELCA SERUM
ALBUMIN PRECURSOR (ALLERGEN FEL D 2)
5. 1.1 × 10 − 4
(nr-Mus-musculus) 48.8 kDa-gi|191765|gb|AAA37190.1| (M16111) alpha-
fetoprotein [Mus musculus]
6. 2.6 × 10 − 3
(nr-Other-Mammalia) 70.8 kDa-gi|1351909|sp|P49065|ALBU_RABIT SERUM
ALBUMIN PRECURSOR
7. 5.8 × 10 − 3
(nr-Homo-sapiens) 71.2 kDa-gi|28590|emb|CAA23753.1| (V00494) reading frame
HSA [Homo sapiens]
8. 2.5 × 10 − 2
(nr-Other-Rodentia) 70.9 kDa-gi|3121749|sp|O35090|ALBU_MERUN SERUM
ALBUMIN PRECURSOR
Sample F6 & G6 Pooled
2. 1.0 × 10 − 9
(nr-Other-Mammalia) 67.8 kDa-gi|3319897|emb|CAA76841.1| (Y17737) albumin
[Canis familiaris]
3. 2.0 × 10 − 7
(nr-Other-Mammalia) 70.6 kDa-gi|1351908|sp|P49064|ALBU_FELCA SERUM
ALBUMIN PRECURSOR (ALLERGEN FEL D 2)
4. 6.2 × 10 − 6
(nr-Other-Mammalia) 93.9 kDa-gi|2507247|sp|P06868|PLMN_BOVIN
PLASMINOGEN PRECURSOR
5. 3.1 × 10 − 3
(nr-Mus-musculus) 48.8 kDa-gi|191765|gb|AAA37190.1| (M16111) alpha-
fetoprotein [Mus musculus]
Sample F7 & G7 Pooled
1. 1.3 × 10 − 43
(nr-Other-Mammalia) 79.9 kDa-gi|2501351|sp|Q29443|TRFE_BOVIN
SEROTRANSFERRIN PRECURSOR (SIDEROPHILIN) (BETA-1-METAL BINDING
GLOBULIN)
2. 5.1 × 10 − 7
(nr-Rattus) 24.9 kDa-gi|92907|pir∥A39684 hemiferrin - rat
3. 5.7 × 10 − 4
(nr-Other-Mammalia) 30.9 kDa-gi|2147092|pir∥I46986 albumin - dog (fragment)
4. 5.7 × 10 − 4
(nr-Mus-musculus) 48.8 kDa-gi|191765|gb|AAA37190.1| (M16111) alpha-
fetoprotein [Mus musculus]
5. 2.9 × 10 − 2
(nr-Other-Primates) 70.5 kDa-gi|1363928|pir∥JC4258 alpha-fetoprotein precursor -
chimpanzee
Sample F12 & G12 Pooled
1. 3.2 × 10 − 7
(nr-Other-Mammalia) 39.5 kDa-gi|231469|sp|P29701|A2HS_SHEEP ALPHA-2-HS-
GLYCOPROTEIN PRECURSOR (FETUIN-A)
Sample H1
1. 4.1 × 10 − 28
(nr-Other-Mammalia) 39.2 kDa-gi|112909|sp|P12763|A2HS_BOVIN ALPHA-2-HS-
GLYCOPROTEIN PRECURSOR (FETUIN-A) (ASIALOFETUIN)
2. 5.2 × 10 − 25
(nr-Other-Mammalia) 39.5 kDa-gi|231469|sp|P29701|A2HS_SHEEP ALPHA-2-HS-
GLYCOPROTEIN PRECURSOR (FETUIN-A)
Sample H2
1. 6.4 × 10 − 28
(nr-Other-Mammalia) 39.2 kDa-gi|112909|sp|P12763|A2HS_BOVIN ALPHA-2-HS-
GLYCOPROTEIN PRECURSOR (FETUIN-A) (ASIALOFETUIN)
2. 3.9 × 10 − 25
(nr-Other-Mammalia) 39.5 kDa-gi|231469|sp|P29701|A2HS_SHEEP ALPHA-2-HS-
GLYCOPROTEIN PRECURSOR (FETUIN-A)
Sample H3
1. 3.0 × 10 − 14
(nr-Other-Mammalia) 39.2 kDa-gi|112909|sp|P12763|A2HS_BOVIN ALPHA-2-HS-
GLYCOPROTEIN PRECURSOR (FETUIN-A) (ASIALOFETUIN)
2. 2.5 × 10 − 7
(nr-Other-Mammalia) 39.5 kDa-gi|231469|sp|P29701|A2HS_SHEEP ALPHA-2-HS-
GLYCOPROTEIN PRECURSOR (FETUIN-A)
Sample H4
1. 6.6 × 10 − 30
(nr-Other-Mammalia) 39.2 kDa-gi|112909|sp|P12763|A2HS_BOVIN ALPHA-2-HS-
GLYCOPROTEIN PRECURSOR (FETUIN-A) (ASIALOFETUIN)
2. 6.0 × 10 − 19
(nr-Other-Mammalia) 39.5 kDa-gi|231469|sp|P29701|A2HS_SHEEP ALPHA-2-HS-
GLYCOPROTEIN PRECURSOR (FETUIN-A)
Sample H5
1. 1.8 × 10 − 21
(nr-Other-Mammalia) 39.2 kDa-gi|112909|sp|P12763|A2HS_BOVIN ALPHA-2-HS-
GLYCOPROTEIN PRECURSOR (FETUIN-A) (ASIALOFETUIN)
2. 5.2 × 10 − 8
(nr-Other-Mammalia) 39.5 kDa-gi|231469|sp|P29701|A2HS_SHEEP ALPHA-2-HS-
GLYCOPROTEIN PRECURSOR (FETUIN-A)
Sample H6
1. 5.7 × 10 − 8
(nr-Other-Mammalia) 39.2 kDa-gi|112909|sp|P12763|A2HS_BOVIN ALPHA-2-HS-
GLYCOPROTEIN PRECURSOR (FETUIN-A) (ASIALOFETUIN)
2. 9.7 × 10 − 3
(nr-Other-Mammalia) 39.5 kDa-gi|231469|sp|P29701|A2HS_SHEEP ALPHA-2-HS-
GLYCOPROTEIN PRECURSOR (FETUIN-A)
Sample H7
1. 7.4 × 10 − 43
(nr-Other-Mammalia) 71.2 kDa-gi|1351907|sp|P02769|ALBU_BOVIN SERUM
ALBUMIN PRECURSOR (ALLERGEN BOS D 6)
2. 3.2 × 10 − 3
(nr-Other-Mammalia) 39.2 kDa-gi|112909|sp|P12763|A2HS_BOVIN ALPHA-2-HS-
GLYCOPROTEIN PRECURSOR (FETUIN-A) (ASIALOFETUIN)
Sample H8
1. 7.6 × 10 − 4
(nr-Other-Mammalia) 39.2 kDa-gi|112909|sp|P12763|A2HS_BOVIN ALPHA-2-HS-
GLYCOPROTEIN PRECURSOR (FETUIN-A) (ASIALOFETUIN)
Note:
see gel image in FIG. 3

1. A composition for enhancing the production of proteoglycan comprising notochord enriched media and/or one or more factors derived from notochord enriched media.

2. A composition according to claim 1 for the treatment of degenerative disease of the chondrocyte matrix.

3. A composition according to claim 1, for the treatment of degenerative disc disease.

4. The composition according to claim 1, wherein the notochord enriched media is obtained from nonchondrodystrophic animals.

5. The composition according to claim 4, wherein the animal is canine, feline or is a rabbit.

6. The composition according to claim 1, wherein the factors derived from notochord enriched media are proteins having a molecular weight in the range of 25 to 220 kilodaltons and a pH in the neutral to acidic range.

7. The composition according to claim 1, further comprising a pharmaceutically acceptable carrier.

8. A method of preparing notochord enriched media comprising:
(a) providing isolated notochord cells; and
(b) culturing the notochord cells in a medium suitable for maintaining the notochord cells.

9. A method of preparing notochord enriched media comprising:
(a) separating a nucleus pulposus from an intervertabral disc of a nonchondrodystrophic animal to provide a total nucleus digest;
(b) separating notochord cells from the total nucleus digest; and
(c) purifying the notochord cells and culturing the notochord cells in media to provide notochord enriched media.

10. The method according to claim 8, wherein the animal is a nonchondrodystrophic canine, feline or is a rabbit.

11. The method according to claim 10, wherein the animal is canine.

12. The method according to claim 9, wherein the notochord cells are separated from the total nucleus digest using a Percoll gradient method.

13. A method of preparing notochord enriched media comprising:
(a) separating a nucleus pulposus from an intervertabral disc of a nonchondrodystrophic animal to provide a total nucleus digest;
(b) separating notochord cells from the total nucleus digest;
(c) mixing notochord cells with alginate;
(d) converting the alginate-containing notochord cells to beads;
(e) culturing the beads on a medium comprising one or more infection control substances and growth factors;
(f) washing the beads to remove the growth factors; and
(g) reculturing beads in media to provide notochord enriched media.

14. A composition for enhancing the production of proteoglycan comprising notochord enriched media prepared using a method according to claim 8.

15. A method for enhancing proteoglycan production comprising administering an effective amount of a composition according to claims 1.

16. The method according to claim 15 for treating degenerative disease of the chondrocyte matrix.

17. The method according to claim 15, for treating degenerative disc disease.

18. The method according to claim 15, wherein the composition is administered percutaneously.

19. The method according to claim 15, wherein the animal is a mammal.

20. The method according to claim 19, wherein the animal is human.

21. A use of a composition according to claim 1 to enhance the production of proteoglycan in a cell or animal in need thereof.

22. A use of according to claim 21 to treat degenerative disease of the chondrocyte matrix.

23. A use of a according to claim 21 to treat degenerative disc disease.

24. A use of a composition according to claim 1 to prepare a medicament to enhance the production of proteoglycan in a cell or animal in need thereof.

25. A use according to claim 24 to prepare a medicament to treat degenerative disease of the chondrocyte matrix.

26. A use of according to claim 24 to prepare a medicament to treat degenerative disc disease.

27. The use according to claim 21, wherein the animal is a mammal.

28. The use according to claim 27, wherein the animal is a human.
